(19) United States
(12) Patent Application Publication
(10) Pub. No.: US 2006/0154239 A1
(43) Pub. Date: Jul. 13, 2006

(54) Detection of protease-resistant prion protein after asymmetric spontaneous interaction

(75) Inventor: Dieter Gassner, Peissenberg (DE)

Correspondence Address: Roche Diagnostics Corporation, Inc., 9115 Hague Road, PO Box 50457, Indianapolis, IN, 46250-0457, US

(73) Assignee: Roche Diagnostics Operations, Inc., Indianapolis, IN (US)

(21) Appl. No.: 11/303,902

(22) Filed: Dec. 16, 2005

Related U.S. Application Data
(63) Continuation of application No. PCT/EP04/06860, filed on Jun. 24, 2004.

(30) Foreign Application Priority Data
Jun. 26, 2003 (DE) DE 10328830.9

(51) Int. Cl. C12Q 1/70; G01N 33/53

(52) U.S. Cl. 435/5; 435/7.1

(57) ABSTRACT

The present invention concerns methods for detecting infectious prion protein with improved sensitivity. For this purpose the heterologous non-pathogenic protease-sensitive prion protein PrPc is added to a sample to be examined and is transformed into protease-resistant prion aggregates by asymmetric spontaneous interaction when the infectious prion protein PrPSc is present in the sample.

### RELATED APPLICATIONS

This application is a continuation of PCT/EP2004/006860 filed Jun. 24, 2004, which claims priority to DE 10328830.9 filed Jun. 26, 2003.

### FIELD OF THE INVENTION

The present invention concerns methods for detecting infectious prion protein with improved sensitivity. For this purpose the heterologous non-pathogenic protease-sensitive prion protein PrPc is added to a sample to be examined which is transformed by asymmetric spontaneous interaction into protease-resistant prion aggregates when the infectious prion protein PrPSc is present in the sample.

### BACKGROUND OF THE INVENTION

Prions are the infectious particles responsible for transmissible spongiform encephalopathies (TSE) such as kuru, variant Creutzfeldt-Jakob disease (vCJD), bovine spongiform encephalopathy (BSE), chronic wasting disease (CWD) and scrapie. The main component of prions is the glycoprotein PrPSc which is a conformationally modified isoform of a normal cell surface protein PrPc (Prusiner, PNAS USA 95, 1363-1383, 1998). The disease-associated prion molecule PrPSc is able to replicate by converting normal PrPc prion molecules.

It is assumed that prion replication occurs according to the “nucleation/polymerization” model which is based on a thermodynamic equilibrium of PrPc and PrPSc in solution (Jarrett and Landsbury, Cell, Vol 73, 1055-1058, 1993; Masel, Jansen and Nowak, Biophys. Chem. 77, 139-152, 1999).

The basis of the model is that the infectious particle is a multimeric, highly ordered aggregate of PrPSc whereas a monomeric PrPSc molecule is unstable and is only stabilized by aggregation with other PrPSc molecules. Hence the rate determining step in replication is the formation of a nucleus which acts to further stabilize PrPSc aggregates.

The PrPSc oligomer elongates itself at the ends of the aggregate to the extent at which new PrPc molecules become attached, converted and incorporated. Hence the kinetics of such a “nucleated prion replication” is limited by the number of PrPSc nuclei that are present in the sample and the potential for PrPc and PrPSc to interact with one another.

Up to now the prion protein PrPSc has been the only marker available for diagnosing diseases of the TSE type. However, the concentration of PrPSc is so low that even in the brain it can only be diagnostically detected in the relatively late phases of a TSE disease. Thus there is only a very limited diagnostic window for detecting TSE diseases.

Therefore attempts have been made to increase the sensitivity of PrPSc detection. Recently a method for increasing the sensitivity of the detection of PrPSc in a sample has been developed based on the above model of prion replication (Saborio et al., Nature 411, 810-813; 2001 and Soto, Biochem. Soc. Trans. 30, 569-574, 2002, WO 02/04954). This method which is referred to as protein misfolding cyclic amplification (PMCA) comprises contacting a sample to be examined with non-pathogenic PrPc whereby small amounts of PrPSc present in the sample interact with the added PrPc to form aggregates, disaggregating aggregates that have formed and determining pathogenic PrPc in the sample. The non-pathogenic PrPc added to the sample is homologous PrPc which is derived from the same species as the sample to be examined. Usually the method consists of several cycles of an experimentally accelerated prion replication. Each cycle consists of two phases. In the first phase very small amounts of PrPSc interact with some PrPc molecules, convert them and thereby induce the growth of PrPSc polymers.

In the second phase these polymers are disintegrated into small fragments by applying ultrasonic waves which exponentially increases the number of potential nuclei in each cycle. The cyclic nature of the method at least theoretically allows as many cycles as are needed until the desired amplification status for the detection of PrPSc is reached.

Finally the aim of the method is to achieve an exponential increase in the number of template units while consuming a PrPc substrate with the aid of a cyclic reaction which consists of the phases aggregation growth and multiplication of the template units.

The PMCA method used in the hamster model has recently been described for other species such as mouse, sheep, goat, cow and humans with the remark that, depending on which species is being used, in particular the ultrasonic strength which needs to be applied for the amplification has to be adapted apparently depending on the state of aggregation of the respective PrPSc polymers (Anderes et al., Poster presentation, Transmissible Spongiform Encephalopathies. New perspectives for prion therapeutics, International Conference, Dec. 1.-3., 2002, Paris, France).

However, the cyclic method for prion amplification appears to be technically susceptible and in the manner in which it is described requires long incubation-sonication cycles.

Wen-Quan and Cashman (J. Biochem. Chem. 277, 43942-43947, 2002) describe that treatment of brain homogenates with acid/guanidinium hydrochloride can result in the formation of PrPSc-like isoforms from non-pathogenic PrPc. The formation of these isoforms can be increased by adding small amounts of the infectious prion protein PrPSc from the brain of Creutzfeldt-Jakob patients.

Lucassen et al. (Biochem. 42, 4127-4135, 2003) describe an in vitro amplification of the protease-resistant prion protein PrPSc by mixing scrapie-infected brain homogenate from the hamster or mouse with homologous normal brain homogenates.

Horiuchi et al. (Proc. Natl. Acad. Sci. USA 97 (2000), 5836-5841) describe interactions between heterologous forms of prion proteins. It was found that the heterologous protease-sensitive prion protein PrPc can bind to the protease-resistant prion protein PrPSc, but is thereby converted into the protease-resistant state only to an extremely slight extent. Furthermore, the presence of the heterologous protease-sensitive prion protein PrPc can interfere with the conversion of the homologous protease-sensitive prion protein PrPc into the protease-resistant PrPSc. On the basis of these findings it was not to be expected that prion protein aggregates that are formed from the protease-resistant prion protein PrPSc and the heterologous protease-sensitive prion protein PrPc would have an adequate protease resistance for a diagnostic detection method.

### SUMMARY OF THE INVENTION

The object forming the basis of the present invention was to provide a simple, rapid and sensitive method for detecting disease-associated or/and infectious prion protein PrPSc in a sample. The inventive solution of this object comprises the steps:

(a) providing a sample to be examined,

(b) adding the heterologous, non-pathogenic protease-sensitive prion protein PrPc,

(c) transforming the added heterologous normal protease-sensitive prion protein PrPc into protease-resistant prion protein aggregates when PrPSc is present in the sample,

(d) incubating with protease and

(e) determining protease-resistant prion protein aggregates in the sample.

The method according to the invention is based on the fact that externally added heterologous, non-pathogenic protease-sensitive prion protein is bound under suitable conditions to PrPSc aggregates in the presence of disease-specific PrPSc aggregates by a self-induced binding reaction between PrPSc and PrPc referred to as an asymmetric spontaneous interaction and surprisingly reaches the state of a diagnostically detectable protease resistance by a binding interaction. The method allows a detection of the infectious prion protein PrPSc with improved sensitivity and a detection of heterologous prion protein isoforms using species-specific prion antibodies.

### BRIEF DESCRIPTION OF THE DRAWINGS

FIG. 1: ICSM 18 (Imperial College London, UK): 500 ng/ml; ICSM 18 reacts with hamster, human and bovine PrP.

FIG. 2: 12F10 (SPIO-BIO, France): 500 ng/ml; 12F10 reacts with human and bovine PrP.

FIG. 3: ICSM 35 (Imperial College London, UK): 500 ng/ml; ICSM 35 reacts with human and bovine PrP and, in comparison with ICSM 18, more strongly with hamster PrP than with bovine PrP.

FIG. 4: 3F4 (Signet, USA): 500 ng/ml; 3F4 reacts with hamster and human PrP but not with bovine PrP in the Western blot.

FIG. 5: 6H4 (Prionics, Switzerland): 500 mg/ml; 6H4 reacts with human and bovine PrP and to a slighter extent with hamster PrP.

FIG. 6: Monoclonal antibody for the Western blot: Antibody L42 (R Biopharm, Germany): 250 ng/ml; L42 reacts with human and bovine PrP but has only a weak cross-reactivity with hamster PrP in the Western blot.

### DESCRIPTION OF THE INVENTION

The object forming the basis of the present invention was to provide a simple, rapid and sensitive method for detecting disease-associated or/and infectious prion protein PrPSc in a sample. The inventive solution of this object comprises the steps:

(a) providing a sample to be examined,

(b) adding the heterologous, non-pathogenic protease-sensitive prion protein PrPc,

(c) transforming the added heterologous normal protease-sensitive prion protein PrPc into protease-resistant prion protein aggregates when PrPSc is present in the sample,

(d) incubating with protease, and

(e) determining protease-resistant prion protein aggregates in the sample.

The method according to the invention is based on the fact that externally added heterologous, non-pathogenic protease-sensitive prion protein is bound under suitable conditions to PrPSc aggregates in the presence of disease-specific PrPSc aggregates by a self-induced binding reaction between PrPSc and PrPc referred to as an asymmetric spontaneous interaction and surprisingly reaches the state of a diagnostically detectable protease resistance by a binding interaction. The method allows a detection of the infectious prion protein PrPSc with improved sensitivity and a detection of heterologous prion protein isoforms using species-specific prion antibodies.

In this connection it is assumed that protease-sensitive low-aggregated PrPSc exists in a PrPSc-positive sample in various states of aggregation of heterogeneous polymer sizes in addition to high-molecular protease-resistant PrPSc aggregates (Tzaban et al., Biochemistry 41, 12868-12875, 2002). This low-aggregated PrPSc can be used by the heterologous PrPc added to the sample as a template for an asymmetric spontaneous interaction which allows the formation of protease-resistant prion protein aggregates.

Under appropriate conditions admixing heterologous PrPc substrate and its attachment/binding to variously aggregated PrPSc aggregates can bring about a protecting binding event for the substrate which results in an increase in the resistance of heterologous PrPc to protease-digestion due to its binding interaction with PrPSc. Moreover, an increase in the state of aggregation due to the binding of heterologous PrPc can apparently increase the protease resistance of the aggregates and consequently also of their components.

Only when PrPSc is present in the sample does this result in the formation of PrPSc/PrPc mixed aggregates which contain heterologous PrPc that has become protease resistant and which can be detected depending on the selection of appropriate species-specific/cross-reacting detection antibodies against prion protein. The detection can be carried out by using known methods such as Western Blot, ELISA etc. for the direct or indirect detection of disease-specific PrPSc (in addition to the PrPSc that is present or exclusive thereof depending on the selected specificity of the prion detection antibodies that are used).

The method results in a considerable increase in the sensitivity of the detection of disease-specific PrPSc especially when a protease-resistant prion protein-induced copolymerization (i.e. binding) of both homologous and particularly heterologous protease-sensitive prion protein occurs progressively in a mixed system (progressive binding events of heterologous PrPc due to PrPc bound in the aggregate that cannot be converted into PrPSc possibly due to the binding of conformationally changed PrPc).

In a first embodiment the method comprises admixing heterologous PrPc substrate (e.g. normal hamster brain homogenate) with a potentially PrPSc-positive or PrPSc-negative template (e.g. BSE-positive or BSE-negative bovine homogenate) under suitable conditions followed by an incubation step for the spontaneous binding interaction of the two heterologous protease-sensitive prion protein PrP forms on a PrPSc template aggregate and subsequent protease digestion.

A binding interaction of heterologous (and homologous) PrPc only occurs in the presence of the PrPSc template whereas heterologous PrPc together with the endogenous homologous PrPc are completely digested by protease in the absence of the PrPSc template.

The heterologous PrPc that is resistant to protease digestion due to binding to the PrPSc template in the aggregate can then be subsequently diagnostically detected by an appropriately selected detection system and used as a sensitive indicator for the disease-specific prion protein PrPSc that is primarily present.

After adding the heterologous protease-sensitive PrPc, the sample is incubated preferably under membrane solubilization conditions in the presence of sphingomyelin/cholesterol-rich detergent-resistant membranes (DRM), so-called lipid rafts or caveolae-like domains (CLDs) (Vey et al., Proc. Natl. Acad. Sci., USA, Vol. 93, 14945-14949, 1996; Baron et al., EMBO J., 21, 1031-1040, 2002) that are required for the binding of PrPc to PrPSc and by means of which PrPc and PrPSc are apparently associated via glycosyl phosphatidyl inositol (GPI) anchors.

Under appropriate conditions, an asymmetric spontaneous binding reaction of PrPc to a PrPSc template takes place in a cell-free binding /converting system due to a PrPc/PrPSc lipid raft—membrane interaction of the heterologous prion protein forms located in the lipid rafts resulting in a relative protease resistance of the heterologous PrPc due to the binding event with the PrPSc aggregates which can be used to detect PrPSc diagnostically.

The diagnostic advantages of the asymmetric spontaneous binding interaction (ASI) can be described as follows:
The method can be simply carried out under almost physiological conditions in for example brain homogenates or other cell-free systems;
Sensitivity of the PrPSc detection is increased due to an increase in the protease resistance of heterologous PrPc after binding to PrPSc;
Possibility of indirectly detecting PrPSc via a heterologous PrPc substrate;
Depending on the selection of detection antibodies, an additive detection of PrPSc/heterologous PrPc or an exclusive heterologous PrPc detection is possible thus lo increasing the sensitivity when detecting PrPSc in tissues and for example cellular blood components such as buffy coat etc. or in other body fluids in which the concentrations of the PrPSc template are very low; and
Compared to a homologous PrPSc/PrPc binding reaction (spontaneous transformation reaction, STR) there is an additional advantage that, depending on the species specificity (at a molecular level on differences in the amino acid sequences of host/inoculum PrP molecules), non-convertible or only partially convertible heterologous PrPc can, in contrast to homologous i.e. convertible PrPc, be detected in the protease-resistant aggregate. Depending on the respective denaturing conditions required to firstly make antibody epitopes accessible in the PrPSc aggregate, the attached non-converted heterologous PrPc can be more easily detached as a monomer and can contribute to the sensitive detection of PrPSc that is primarily present after a protease digestion.

Step (a) of the method comprises the provision of a sample to be examined. The sample can be derived from tissue or body fluids that may contain prion protein such as the brain, nervous tissue or the lymphoreticular system e.g. blood or blood components. The samples are usually provided in the form of homogenates which contain a lipid raft preserving detergent e.g. a non-ionic detergent such as Triton X100. In particular bovine or human samples are preferably free of ionic detergents such as SDS. Samples of body fluids such as blood, cellular blood components, buffy coats etc. can be prepared by concentrating cells containing prion protein e.g. by isolating lymphocytes and other mononuclear cells from anticoagulated whole blood (e.g. Accuspin system Histopaque 1077, Sigma Diagnostics). The sample is preferably provided under essentially physiological conditions e.g. pH 6-8 and salt concentrations corresponding to 50 to 500 mmol/l NaCl. A protease inhibitor or a combination of protease inhibitors (e.g. Protease-Inhibitor Cocktail complete, Roche Diagnostics) is advantageously added to the sample in order to activate endogenous proteases present in the sample. After the homogenization the sample is preferably used directly or freshly i.e. without prior freezing.

Step (b) of the method according to the invention comprises adding the heterologous non-pathogenic protease-sensitive prion protein PrPc to the sample to be examined. The ratio of added heterologous prion protein to the prion protein present in the sample is 1:99 to 99:1, particularly preferably 10:90 to 90:10 where in general homogenates are used with essentially equal concentrations in each case e.g. 10%.

The term “heterologous” in the sense of the present invention means that the normal prion protein PrPc of a foreign species, preferably of a foreign genus is added to the sample. The addition of rodent PrPc e.g. hamster PrPc or mouse PrPc to a bovine sample, a sheep sample or a human sample is for example particularly preferred. It is also preferred to add human PrPc to a bovine sample or to add bovine or sheep PrPc to a human sample. The heterologous material, preferably a PrPc homogenate which is added to the sample is particularly preferably a fresh homogenate which has not been frozen after the homogenization.

Step (c) of the method according to the invention preferably comprises an incubation of the sample under conditions in which an asymmetric spontaneous interaction of the heterologous protease-sensitive prion protein PrPc takes place with the infectious prion protein PrPSc present in the sample to form protease-resistant prion protein aggregates. This asymmetric spontaneous interaction comprises an attachment of the heterologous non-pathogenic prion protein PrPc to the infectious prion protein PrPSc present in the sample.

The sample is preferably incubated at a temperature of 20-55° C., in particular of 35-50° C. The incubation is carried out for a period which is sufficient to achieve an effective increase in sensitivity. The incubation period is preferably at least 10 min e.g. 10-240 min and particularly preferably 15-120 min. A deaggregation step can optionally be carried out before step (c) in which high-molecular PrPSc aggregates are deaggregated into low-molecular aggregates. Such a step can for example comprise a single ultrasonic treatment.

The method according to the invention can additionally comprise one or more additional amplification cycles i.e. one or more successive ultrasonic/incubation cycles e.g. corresponding to the PMCA method.

On the other hand, when an immunoassay was used for the analysis it was found that a longer incubation period or an amplification are not necessary to achieve a high sensitivity in certain embodiments of the invention.

The protease according to step (d) of the method according to the invention is selected such that it is able to cleave the non-pathogenic, homologous prion protein PrPc present in the sample and the added heterologous prion protein PrPc into a monomeric form whereas added heterologous PrPc in the form of aggregates with PrPSc is substantially resistant to cleavage. An example of a suitable protease is proteinase K. Proteinase K is particularly preferably used at a concentration of 50-100 μg/ml.

Step (e) of the method according to the invention comprises the determination of the protease-resistant prion protein PrPSc in the sample. This determination can be carried out qualitatively or/and quantitatively by all methods known in the prior art. Examples of suitable methods are immunological methods in which pathogenic prion protein is determined by reaction with a specific antibody.

In a particularly preferred embodiment the prion protein is determined by a Western blot. For this the sample is electrophoretically separated under denaturing conditions e.g. by SDS-PAGE and the proteins contained therein are blotted onto a suitable membrane e.g. a nitrocellulose or polyvinylidene fluoride (PVDF) membrane. The prion protein is then made visible on the membrane by reaction with polyclonal or monoclonal anti-prion antibodies which can be directly labelled or can be detected by a labelled secondary antibody. In this connection an enzymatic label is preferred while using a detectable substrate e.g. a chemiluminescent substrate. Commercially available anti-prion antibodies are mentioned in the examples.

On the other hand, the determination can also be carried out by means of an immunoassay in which the sample, without prior electrophoretic separation, is contacted with suitable detection reagents. The immunoassay is preferably carried out as a sandwich assay using a solid phase antibody against the prion protein e.g. a biotinylated and labelled antibody e.g. an enzyme-labelled antibody. The detection is particularly preferably carried out by means of a sandwich ELISA in which one or more anti-prion antibodies are used and an enzyme-antibody conjugate is used as a labelled secondary antibody together with a detectable enzyme substrate.

In order to detect the protease-resistant aggregates that are formed when infectious PrPSc is present in the sample, one can, as already mentioned, use antibodies which recognize the prion protein either species- or genus-specifically or independently of species or genus. Combinations or mixtures of two or more such antibodies can also be used.

Antibody combinations comprising antibodies that are specific for rodents e.g. hamster or/and bovine prion protein or antibodies that are specific for bovine or/and human prion protein are used in a particularly preferred embodiment of the method according to the invention.

It is intended to further elucidate the invention by the following examples.

### Specific Embodiments

Asymmetric Spontaneous Interaction Between Heterologous PrP Forms in the Bovine/Hamster System

In this example it is shown that an asymmetric spontaneous interaction between heterologous PrP forms can take place e.g. hamster PrPc can bind to bovine PrPSc aggregates. This is compared with a spontaneous transformation reaction between homologous PrP forms e.g. the binding of bovine PrPc to bovine PrPSc aggregates in a cell-free binding/conversion system.

Sample Preparation

BSE-positive bovine brain homogenate (20% homogenate in 10% sucrose) obtained from the obex region of the medulla oblongata (VLA case 99/00946) was diluted 100-fold with ice-cold:

(a) normal bovine brain homogenate (homologous system) obtained from the obex region of the medulla oblongata of a healthy cow as a 10% homogenate in PBS buffer containing 0.5% Triton X100 and protease inhibitor cocktail complete (Roche Diagnostics) or

(b) normal brain homogenate of the Syrian hamster (heterologous system) as a 10% homogenate in PBS buffer containing 0.5% Triton X100 and protease inhibitor cocktail complete (Roche Diagnostics).

The homogenates were prepared completely using a Ribolyser tissue homogenizer (Hybaid, UK) and green tubes containing ceramic beads from Hybaid (UK). After homogenizing normal brain samples in PBS buffer containing the protease inhibitor cocktail complete, Triton X100 was added to a final concentration of 0.5% and the homogenates were solubilized for 15 min at 25° C. while shaking. The normal brain homogenates were then placed in an ice bath and mixed with BSE brain homogenate as stated above. 200 μl aliquots were then subjected to the treatment procedures described in the following where the 0 min samples were kept in an ice bath.

Immediately after the incubation all samples were digested for 60 min at 37° C. with proteinase K (final concentration 100 μg/ml). The reaction was stopped by adding PMSF at a final concentration of 40 mM. Then the samples were divided into aliquots and analysed.

Normal hamster sample (digestion control): sample 2;

Normal bovine sample (digestion control): sample 9;

Homologous System (Bovine PrPSc/Bovine PrPc):

Incubation at 47° C. for 0/15/30/60 min while shaking at 500 rpm (Eppendorf shaker); samples 10-13;

Indirect sonication (1 pulse of 15 sec) using a microsonicator (Bandelin Electronik, Sono plus, Berlin) equipped with a Becher resonator (BR30) and a sample holding device (EH3) followed by incubation at 47° C. for 0/15/30/60 min while shaking at 500 rpm (Eppendorf shaker): samples 14-17;

Heterologous System (Bovine PrPSc/Hamster PrPc):

Incubation at room temperature for 0/60 min while shaking at 500 rpm (Eppendorf shaker): samples 3,4;

Incubation at 47° C. for 0/60 min while shaking at 500 rpm (Eppendorf shaker): samples 5, 6;

Indirect sonication (1 pulse of 15 sec) using a microsonicator (Bandelin Electronik, Sono plus, Berlin) equipped with a Becher resonator (BR30) and a sample holding device (EH3) followed by incubation at 47° C. for 0/60 min while shaking at 500 rpm (Eppendorf shaker): samples 7, 8.

After the stated incubation periods the samples were returned to the ice bath and subsequently digested in parallel with proteinase K.

Western Blot Analysis

An SDS-PAGE was carried out under reducing conditions (5 min at 95° C.) by mixing the sample with 2× SDS sample buffer followed by an electroblot onto a PVDF membrane. The membranes were treated with the Dig block and washing buffer kit (Roche Diagnostics) and incubated for 1 h with a set of various species-specific monoclonal anti-prion antibodies (as stated in the following) followed by a 30 min incubation with a sheep anti-mouse IgG-alkaline phosphatase conjugate (40 mU/ml) Fab fragment (Roche Diagnostics). The reactivity on the membrane was developed by using a CDP-Star chemiluminescent substrate followed by visualization (about 10 min) with a LumiImager system (Roche Diagnostics).

Monoclonal Antibody for the Western Blot:

Antibody L42 (R Biopharm, Germany): 250 ng/ml; L42 reacts with human and bovine PrP but has only a weak cross-reactivity with hamster PrP in the Western blot (FIG. 6).

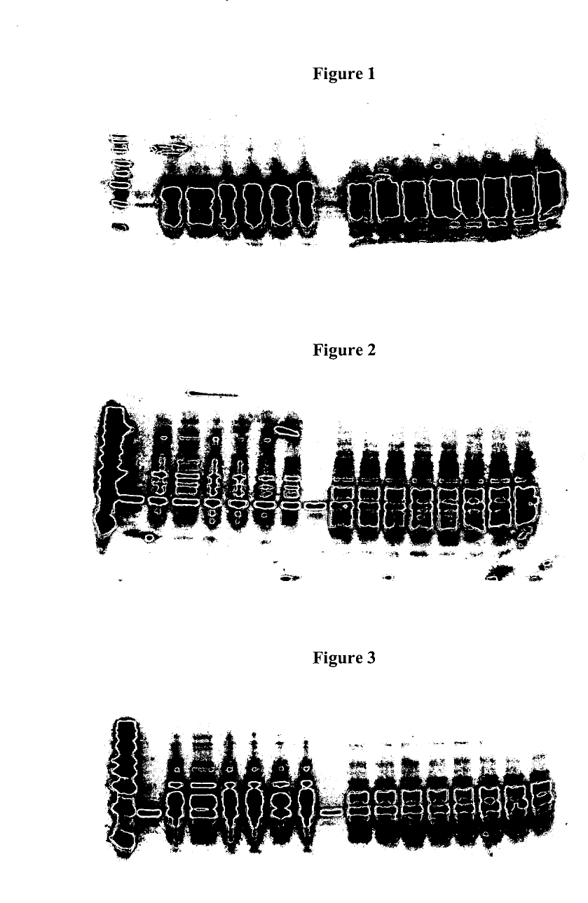

ICSM 18 (Imperial College London, UK): 500 ng/ml; ICSM 18 reacts with hamster, human and bovine PrP (FIG. 1).

ICSM 35 (Imperial College London, UK): 500 ng/ml; ICSM 35 reacts with human and bovine PrP and, in comparison with ICSM 18, more strongly with hamster PrP than with bovine PrP (FIG. 3).

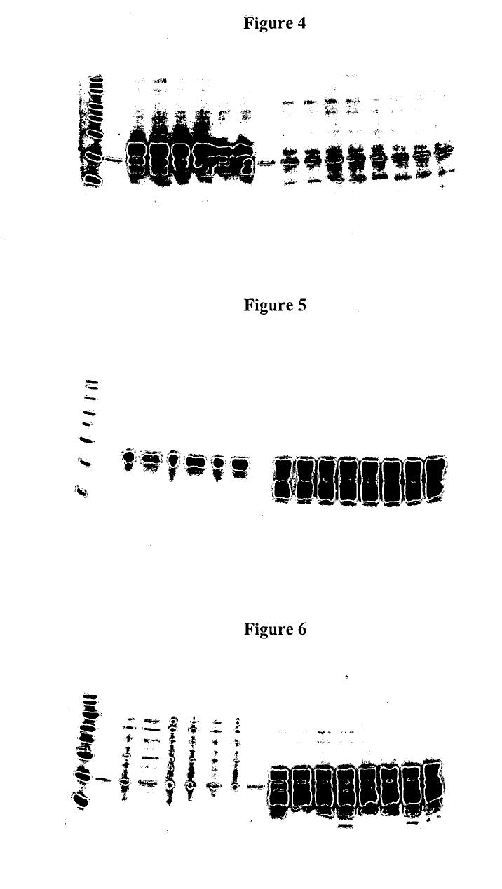

3F4 (Signet, USA): 500 ng/ml; 3F4 reacts with hamster and human PrP but not with bovine PrP in the Western blot (FIG. 4).

12F10 (SPIO-BIO, France): 500 ng/ml; 12F10 reacts with human and bovine PrP (FIG. 2).

6H4 (Prionics, Switzerland): 500 mg/ml; 6H4 reacts with human and bovine PrP and to a slighter extent with hamster PrP (FIG. 5).

The results of samples 2-17 in the Western blot are shown in FIGS. 1-6. Proteinase K-resistant bovine prion bands of <30 kD (double glycosylated, single glycosylated and unglycosylated bands typical for PrPSc) were found in homologous bovine PrPSc/bovine PrPc systems (samples 10-17) depending on the specificity of the antibody that was used (cf. ICSM 18, 12F10, 6H4 and L42). The band pattern at about 35 kDa must be interpreted as bound bovine PrPc that has not yet been converted (cf. samples 10-17).

On the other hand proteinase K-resistant hamster prion bands (about 35 kDa) presumably consisting of hamster PrPc can be recognized in the heterologous bovine PrPSc/hamster PrPc systems i.e. in systems in which a heterologous PrPc binding can take place (cf. antibody reactivity pattern ICSM 18, 35, 3F4 and 6H4, samples 3-8). Depending on the species differences in the prion protein amino acid sequences and the antibody epitope recognition on the prion protein, one can find a limited conversion process (cf. ICSM 18 recognition pattern, samples 3-8) even with the glycoform distribution and the apparent molecular masses of the PrPSc molecules formed in the heterologous system which correspond to the newly converted PrPSc molecules in the homologous system (only visualized as a consequence of the prion protein recognition pattern of the antibody ICSM 18). Digestion controls of normal hamster brain/bovine brain homogenates (used for the dilution experiment) resulted in no protease-resistant PrP bands (samples 2 and 9). A certain cross-reactivity of the second antibody in the Western blot with proteinase K is manifested by the presence of a band of about 30 kDa (samples 2, 9, 3-8, 10-17).

Magic Mark Western Standard (Invitrogen) was used as a molecular weight standard with bands of 120, 100, 80, 60, 50, 40, 30 and 20 kDa (in each case sample 1).

ELISA Analysis

The samples were mixed with 2× guanidinium HCl denaturation buffer (7.6 M guanidinium HCl, final concentration 3.8 M guanidinium HCl) for 15 min at room temperature while shaking at 450 rpm to expose antibody-reactive epitopes. 40 μl of the samples were then added to 200 μl incubation buffer which contained an antibody conjugate mixture of ICSM 35 IgG-biotin (1 μg/ml)/ICSM 18 Fab′-POD polyconjugate (100 mU/ml) and they were incubated for 2 h at 25° C. while shaking (400 rpm) in a microtitre plate (Biocoat, Germany) coated with thermo-BSA-streptavidin. The plates were washed with PBS/0.05% Tween 20 and the immobilized immune complexes were detected by using the substrate TMB (200 μl, 5 min). The enzyme reaction was stopped by adding 50 μl stop solution (2 M H₂SO₄). The OD values were determined with a two-wavelength photometer at 450 nm and a reference wavelength of 690 nm (blank value=0.017).

The results are shown in the table below.

| Sample | 1 | 2 |
|---|---|---|
| 2 | 0.1050 | 0.0940 |
| 3 | 2.0460 | 1.9860 |
| 4 | 1.8970 | 1.9320 |
| 5 | 1.9730 | 1.9830 |
| 6 | 1.8630 | 1.9180 |
| 7 | 1.4550 | 1.4790 |
| 8 | 1.4090 | 1.4680 |
| 9 | 0.0730 | 0.0770 |
| 10 | 0.7600 | 0.8020 |
| 11 | 0.7570 | 0.7680 |
| 12 | 0.7550 | 0.8200 |
| 13 | 0.8050 | 0.8030 |
| 14 | 0.7120 | 0.7530 |
| 15 | 0.6480 | 0.7430 |
| 16 | 0.6870 | 0.7710 |
| 17 | 0.7820 | 0.7390 |

Proteinase K-resistant bovine PrP aggregates are formed in the homologous bovine PrPSc/bovine PrPc system independently of the incubation period (0-60 min) under the experimental conditions used (cf. samples 10-13 and 14-17).

Sample 9 is a normal bovine brain homogenate (PrPc digestion control) which was used as a PrPc source in the homologous dilution experiment.

Proteinase K-resistant aggregates are recognizable in the heterologous bovine PrPSc/hamster PrPc system which are formed independently of the incubation period/temperature under the experimental conditions used (cf. samples 3/4, 5/6, 7/8). The values in the heterologous bovine PrPSc/hamster PrPc system are surprisingly much higher than in the homologous bovine PrPSc/bovine PrPc system.

Sample 2 is a normal hamster brain homogenate (PrPc digestion control) which was used as a PrPc source in the heterologous dilution experiment. A decrease in the OD value in the ELISA system can occur after ultrasonic treatment in the heterologous bovine PrPSc/hamster PrPc system (ELISA samples 7, 8; decrease of about 25%), compared with samples without ultrasonic treatment (ELISA samples 5, 6). On the other hand this decrease does not occur in the homologous bovine PrPSc/bovine PrPc system (cf. ELISA samples 10-13 and samples 14-17).

What is claimed is:

1. A method for detecting the presence of prion protein PrPSc in a sample obtained from an animal, said method comprising:
(a) contacting said sample with heterologous protease-sensitive prion protein PrPc;
(b) incubating said sample under conditions suitable for the spontaneous binding of the heterologous protease-sensitive prion protein PrPc to prion protein PrPSc present in the sample;
(c) adding a protease to the sample; and
(d) screening for the presence of protease-resistant prion protein aggregates in the sample.

2. The method of claim 1 wherein the sample is obtained from cattle, mice, hamsters, sheep, goats or humans.

3. The method of claim 2 wherein the heterologous protease-sensitive prion protein PrPc is derived from an animal of a different species than said sample.

4. The method of claim 2 wherein the heterologous protease-sensitive prion protein PrPc is derived from an animal of a different genus than said sample.

5. The method of claim 1 wherein the heterologous protease-sensitive prion protein PrPc is derived from a rodent species and the sample is derived from cattle, sheep or humans.

6. The method of claim 1 wherein the heterologous protease-sensitive prion protein PrPc is derived from humans and the sample is derived from cattle or sheep

7. The method of claim 2 wherein the heterologous protease-sensitive prion protein PrPc is derived from cattle or sheep and the sample is derived from humans.

8. The method of claim 1 wherein the sample comprises a tissue homogenate and a non-ionic detergent.

9. The method of claim 8 wherein the incubation step (b) comprises incubating the sample in the presence of the heterologous protease-sensitive prion protein PrPc at a temperature of about 20° C. to about 55° C. for about 15 to about 120 minutes.

10. The method of claim 9 further comprising a step of deaggregating PrPSc present after step (b) and then repeating step (b) prior to the step of adding the protease to the sample.

11. The method claim 4, wherein the sample is derived from brain or nerve tissue.

12. The method claim 4, wherein the sample is derived from fluids isolated from the lymphoreticular system.

13. The method of claim 1 wherein said protease is proteinase K.

14. The method of claim 1 wherein said screening step comprises immunologically detecting the presence of PrPSc in the sample.

15. A method for detecting prion protein PrPSc in a sample, said method comprising the steps:
(a) providing a sample to be examined;
(b) adding heterologous protease-sensitive prion protein PrPc to said sample;
(c) transforming the added heterologous protease-sensitive prion protein PrPc into protease-resistant prion protein aggregates when PrPSc is present in the sample;
(d) adding a protease to said sample; and
(e) detecting protease-resistant prion protein aggregates in the sample.

16. The method of claim 15 wherein the sample is obtained from cattle, mice, hamsters, sheep, goats or humans.

17. The method of claim 16 wherein the sample is derived from tissue or body fluids such as brain, nervous tissue or the lymphoreticular system.

18. The method of claim 17 wherein the sample comprises a cell-free homogenate and a non-ionic detergent.

19. The method of claim 15 wherein the heterologous protease-sensitive prion protein PrPc is derived from an animal of a different genus than said sample.

20. The method of claim 19 wherein the heterologous protease-sensitive prion protein PrPc is added as a cell-free homogenate of animal tissue.

21. The method of claim 20 wherein the transforming step (c) comprises incubating the sample in the presence of the heterologous protease-sensitive prion protein PrPc at a temperature of about 20° C. to about 55° C. for at least 10 minutes.

22. The method of claim 21 wherein the protease is proteinase K added to a concentration of about 50 to about 100 μg/ml.

23. The method of claim 19 wherein detection step (e) comprises Western blot analysis or an immunoassay.

24. The method of claim 21 wherein detection step (e) provides a quantitative measurement of PrPSc present in the sample.

25. A method for diagnosing a TSE (transmissible spongiform encephalopathy) disease, said method comprising the steps of:
(a) obtaining a biological sample from an animal;
(b) contacting said sample with heterologous protease-sensitive prion protein PrPc;
(c) incubating said sample under conditions suitable for the spontaneous binding of the heterologous protease-sensitive prion protein PrPc to prion protein PrPSc present in the sample;
(d) adding a protease to the sample; and
(e) screening for the presence of protease-resistant prion protein aggregates in the sample, wherein detection of protease-resistant prion protein aggregates is diagnostic for a TSE disease.

26. The method of claim 25 wherein the animal is selected from the group consisting of humans and farm animals.
